(19) United States
(12) Patent Application Publication
(10) Pub. No.: US 2017/0333242 A1
(43) Pub. Date: Nov. 23, 2017

(54) ORTHOTIC SPLINT

(71) Applicant: Saebo, Inc., Charlotte, NC (US)

(72) Inventors: Henry B. Hoffman, Charlotte, NC (US); Ian D. Kovacevich, Carlsbad, CA (US)

(21) Appl. No.: 15/160,252

(22) Filed: May 20, 2016

(51) Int. Cl. A61F 5/01; A61F 5/042

(52) U.S. Cl. CPC A61F 5/0118; A61F 5/0113; A61F 5/042

(57) ABSTRACT

A dynamic splint for use with a user's hand, including a hand support section, at least one strut including a base portion disposed adjacent the hand support section and a body portion extending outwardly therefrom to a distal end, the body portion defining at least one recess, and at least one tensioner including a proximal end, a distal end configured to be secured to a digit of the user's hand, an elongated body extending therebetween, and at least one node disposed on the elongated body, wherein the at least one node is selectively received in the at least one recess in a press-fit, and the elongated body of the at least one tensioner has flexural strength to resist bending of the digit.

### FIELD OF THE INVENTION

The present invention generally relates to orthoses and, in particular, to dynamic splints.

### BACKGROUND OF THE INVENTION

Many people suffering upper motor neuron injuries from stroke, cerebral palsy, brain injury, etc., have one, or both, of upper and lower extremity impairments. With regard to upper extremity impairments, many have some shoulder and elbow movement, but are unable to extend their wrist or fingers to grasp an object. This is usually due to hypertonicity, described in U.S. Pat. No. No. 5,807,293 to Wedge, Jr., issued Sep. 15, 1998, as a condition where the flexor or extensor muscles in the upper extremities are spastic and resist positioning. Similarly, with regard to lower extremity impairments, it is common to have control of the upper and lower leg, but the inability to maintain the foot in a position that is substantially perpendicular to the lower leg. This condition is commonly referred to as “foot drop.” In addition to neurological injuries, many clients may have physical impairments resulting from orthopedic conditions (i.e., cervical, back, fractured humerus). For example, injuries to the spinal cord may lead to impaired hand function or foot drop. Dynamic splints can be used to offer slight resistance to hold joints in certain positions. An effective dynamic splint designed to be used for mobility and function must offer enough force to support the affected limb functionally, but also allow sufficient assistance as needed. Such a dynamic splint is disclosed and described, for example, in U.S. Pat. No. 7,001,352 to Farrell et al., issued Feb. 21, 2006. Embodiments of the present invention present yet additional, alternative designs for a dynamic splint.

### SUMMARY OF THE INVENTION

One embodiment of a dynamic splint in accordance with the present disclosure includes a hand support section adapted to be releasably secured to a hand of a user, at least one strut including a base portion disposed adjacent the hand support section and a body portion extending outwardly therefrom to a distal end, the body portion defining at least one recess disposed between its proximal end and its distal end, and at least one tensioner including a proximal end, a distal end configured to be secured to a digit of the user's hand, an elongated body extending therebetween, and at least one node disposed on the elongated body. The at least one node is selectively received in the at least one recess in a press-fit, and the elongated body of the at least one tensioner has flexural strength to resist bending of the digit secured to the distal end of the at least one tensioner when the digit is flexed from an extended position toward a flexed position.

Another embodiment of a dynamic splint in accordance with the present disclosure includes a support section configured to be releasably secured to a first portion of an extremity of the user, the support section defining at least one recess therein, and at least one tensioner including a proximal end, a distal end configured to be secured to a second portion of the extremity of the user, an elongated body extending therebetween, and at least one node disposed on the elongated body. The at least one node is selectively received in the at least one recess in a press-fit, and the elongated body of the at least one tensioner has flexural strength to resist bending of the second portion of the extremity secured to the distal end of the at least one tensioner relative to the first portion of the extremity.

Another embodiment of a dynamic splint in accordance with the present disclosure includes a hand support section adapted to be releasably secured to the hand of the user, at least one strut including a base portion disposed adjacent the hand support section and a body portion extending outwardly therefrom to a distal end, the body portion defining a channel extending along its body portion, and at least one tensioner including a proximal end, a distal end configured to be secured to a digit of the user's hand, and an elongated body extending therebetween, wherein a portion of the at least one tensioner is selectively received in the channel in a press-fit, and the elongated body of the at least one tensioner has flexural strength to resist bending of the digit secured to the distal end of the at least one tensioner when the digit is flexed from an extended position toward a flexed position.

In addition to the aforementioned aspects and features of the present invention, it should be noted that the present invention further includes the various possible combinations of such aspects and features. Examples of such combinations are illustrated in the detailed description set forth below.

### BRIEF DESCRIPTION OF THE DRAWINGS

One or more preferred embodiments of the present invention now will be described in detail with reference to the accompanying drawings, which are for the purpose only of illustrating embodiments of the invention and are not intended to be to scale:

FIG. 1 is a top perspective view of a dynamic splint in accordance with an embodiment of the present disclosure;

FIG. 2 is a top perspective view of the dynamic splint as shown in FIG. 1 on a user's hand;

FIG. 3A is a top perspective view of a strut the dynamic splint shown in FIG. 1;

FIG. 3B is a top view of the strut shown in FIG. 3A;

FIG. 4 is a partial, top perspective view of the dynamic splint shown in FIG. 1, enlarged to show greater detail;

FIG. 5 is a bottom perspective view of a hand support section and struts of the dynamic splint shown in FIG. 1;

FIG. 6A is a front plan view of an alternate embodiment of a dynamic splint in accordance with the present disclosure;

FIG. 6B is a plan view of an attachment strap of the dynamic splint shown in FIG. 6A;

FIG. 6C is a plan view of a tensioner of the dynamic splint shown in FIG. 6A; and

FIG. 7 is a perspective view of the splint shown in FIG. 6A on a user's foot.

The accompanying drawings, which are incorporated in and constitute a part of this specification, illustrate one or more embodiments of the invention and, together with the description, serve to explain the principles of the invention.

### DETAILED DESCRIPTION OF THE INVENTION

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at lease one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Additionally, several terms such as “dorsal,” “volar,” “radial,” and “ulnar” are used herein with reference to features of the human hand and foot. Indeed, descriptions herein of one or more illustrated embodiments of the invention sometimes are made with such terms that may imply that the embodiment is disposed on a forearm and hand, or leg and foot. Use of such terms of reference is made herein in order to facilitate an understanding of the invention, and the forearm and the hand are not considered in such embodiments to be actual elements of the invention.

Moreover, for the purpose of interpreting these terms of reference, the reader should consider a forearm and open hand resting palm-side down upon a planar desktop, with the forearm and palm generally contacting the desktop, and with the fingers and thumb generally straight and resting their lengths on the desktop. The volar sides of the forearm, wrist, hand, and fingers are generally disposed toward and contact the desktop. Thus, the fingerprints generally are found on the volar sides of fingertips. The dorsal sides of the forearm, wrist, hand, and fingers generally face in opposite direction to the volar sides of the forearm, wrist, hand, and fingers. These dorsal sides thus would be generally oriented away from the desktop. For example, fingernails generally grow from the dorsal sides of the fingers. The side of the hand from which the thumb depends defines the radial side of the forearm, wrist, and hand. In contrast, the side of the hand opposing the radial side defines the ulnar side of the forearm, wrist, and hand. For example, the fourth finger from the thumb of the hand, generally the smallest finger often called the “pinkie” finger, depends from the ulnar side of the hand. In view of these clarifications, these terms of reference are unambiguous and are well-defined with regard to essentially any hand or wrist, including both the left hand and right hand.

Regarding the views of the figures, dorsal views herein refer to views directed toward dorsal sides. For example, a dorsal view of a hand shows the dorsal side of the hand, which side is sometimes called the back of the hand. Similarly, a radial view of a hand generally would include a showing of the thumb, a volar view of a hand generally would include a showing of the palm, and an ulnar view of a hand generally would include a showing of the fourth finger from the thumb.

Regarding planes and axes, volar-dorsal planes are generally perpendicular to radial-ulnar planes, and the forearm generally defines a longitudinal axis. The reader should consider again the forearm and hand resting palm-side down on a planar desktop, particularly when the hand and forearm are comfortably aligned and the fingers are extended straight and held tightly together. In this disposition of the forearm and hand, the plane of the desktop defines a radial-ulnar plane; a longitudinal axis is defined along the length of the forearm; and the four fingers of the hand extend generally parallel to the longitudinal axis. Furthermore, rotation of a radial-ulnar plane by ninety degrees about the longitudinal axis produces a volar-dorsal plane. For example, when a postcard is slipped between adjacent fingers such that an edge of the postcard abuts the desktop and is held parallel to the longitudinal axis, and such that the postcard stands vertically and ninety degrees from the plane of the desktop, the postcard defines a volar-dorsal plane.

Furthermore, terms of reference such as “phalanx,” “phalange,” and “interphalangeal joint,” which terms are well-known and are found in the prior art, may be used herein with reference to the skeletal anatomy of the human hand. Indeed, descriptions herein of one or more illustrated embodiments of the invention sometimes are made with such terms that may imply that the embodiment is disposed on or abuts the hand. Use of such terms of reference is made herein in order to facilitate an understanding of the invention while the hand and portions thereof are not necessarily considered in such embodiments to be actual elements of the invention.

Nonetheless, for the purpose of interpreting these terms of reference, reference is herein made to the fourth figure of U.S. Pat. No. 5,676,157 to Kramer, which issued on Oct. 14, 1997 (the “Kramer patent”). In the fourth figure of the Kramer patent, which figure is hereby incorporated herein by reference, the skeletal anatomy of a human hand is illustrated wherein particular bones and joints defined therebetween are identified. For the purpose of interpreting terms of reference as used herein, the fourth figure of the Kramer patent may be regarded as a dorsal view of a right hand. As shown and as is commonly known, five digits, including a thumb and four fingers, depend from the hand. The three bones of any one of the four fingers, disposed in increasing distance from the hand, are referred to as: the proximal phalange (or proximal phalanx); the middle phalange (or middle phalanx); and the distal phalange (or distal phalanx). A section of a finger may be referred to herein with regard to a particular phalange without ambiguity in that such a section would include the particular bony phalange and the flesh of the finger about the phalange. For example, in typing or in entering data using a keyboard, distal phalange sections of the fingers generally abut and actuate keys of the keyboard without regard to whether distal phalange bones, which are generally surrounded by the flesh of the fingers, ever directly contact the keyboard.

With regard to joints, for each of the four fingers illustrated in the fourth figure of the Kramer patent, a proximal interphalangeal joint is defined between the proximal phalange and the middle phalange, and a distal interphalangeal joint is defined between the middle phalange and the distal phalange. The thumb, however, having less joints than each of the four fingers, generally includes an interphalangeal joint, indicated in the fourth figure as “THUMB IP,” defined between a proximal phalange (or proximal phalanx) and a distal phalange (or distal phalanx). Thus, any recitation herein relating to the “last joint” or “distal joint” of a digit relates equally to any distal interphalangeal joint of a finger and to any interphalangeal joint of a thumb regarding either a left hand or a right hand.

Turning now to the drawings of the present application, preferred embodiments of dynamic splints of the present invention are illustrated and are described in detail below. It should be furthermore understood that the views found in the accompanying drawings relate to dynamic splints for both a right forearm, wrist, and hand, and a right lower leg, ankle and foot. Nevertheless, the accompanying drawings and the descriptions herein relate equally as well to dynamic splints for a left forearm, wrist, and hand, and a left lower leg, ankle and foot, when a mirror image of the various drawings is considered.

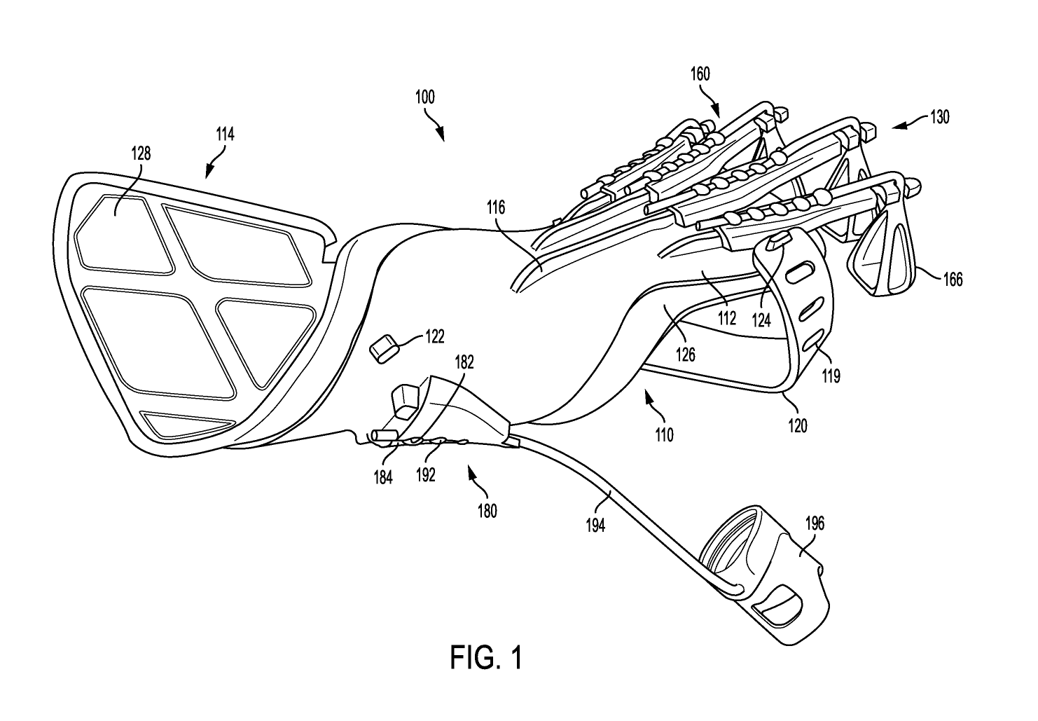

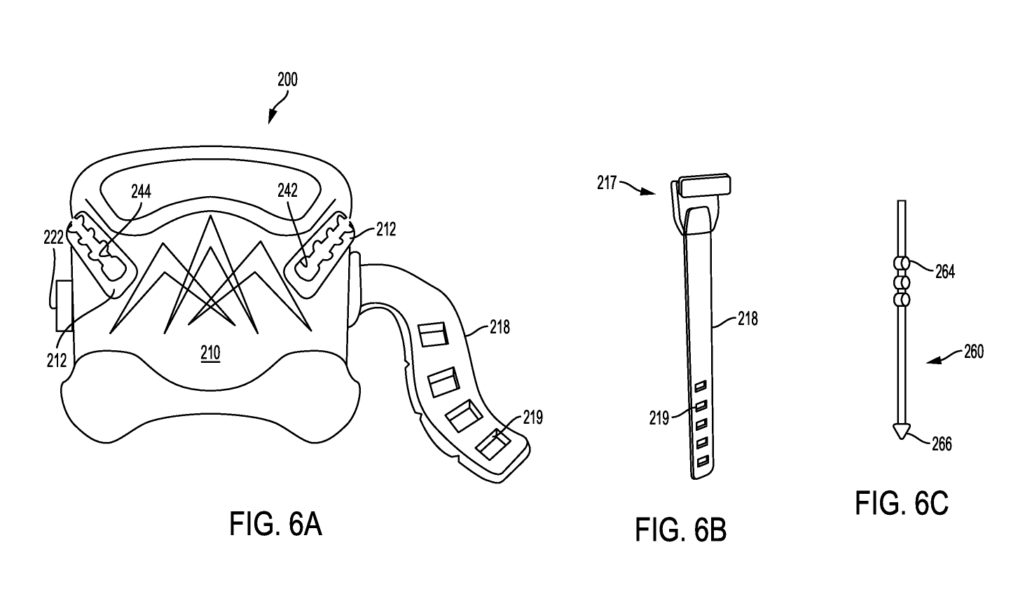

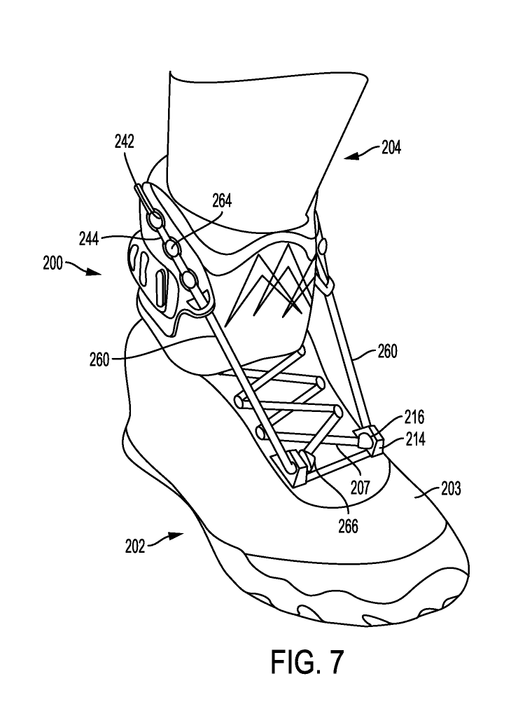

Each disclosed embodiment comprises a dynamic splint for the positioning and exercise of a neurologically impaired extremity. For example, the first disclosed embodiment (FIGS. 1 through 5) is for use with an upper extremity including the wrist, hand, and fingers, and is specifically directed toward a dynamic splint that exercises a rehabilitating hand by providing resistance to the hand's fingers and thumb. The first embodiment is especially useful for returning the fingers and thumb to an open or extended position after a grasping motion has been carried out and, specifically, is used to hold the user's impaired wrist, hand and fingers generally in an extended position, with the thumb in palmer abduction. This position places the impaired hand in the functional position for grasping. The first embodiment thus allows a neurologically impaired upper extremity, including the hand, to work on repetitive grasp-and-release activities while participating in task-specific arm training. The first embodiment further has a dynamic characteristic that offers varying degrees of substantial resistance to the digits. Additionally, the second embodiment (FIGS. 6A through 7) is for use with a lower extremity including the lower leg, ankle and foot, and is specifically directed toward a dynamic splint that exercises a rehabilitating foot.

Referring now to the figures, an embodiment of a dynamic splint 100 in accordance with the present disclosure is illustrated in FIGS. 1 through 5, the dynamic splint being configured for use in rehabilitating a user's hand 102. The present embodiment includes a hand support section 110 in which a platform 112 and a forearm support section 114 are integrally formed. A plurality of stiffening ribs 116 extend along the top surface of platform 112 to lend rigidity to hand support section 110. Further, hand support section 110 includes a wrist strap 118 and a palm strap 120 that are releasably secured at their distal ends to attachment posts 122 and 124, respectively, when donning the hand splint 100.

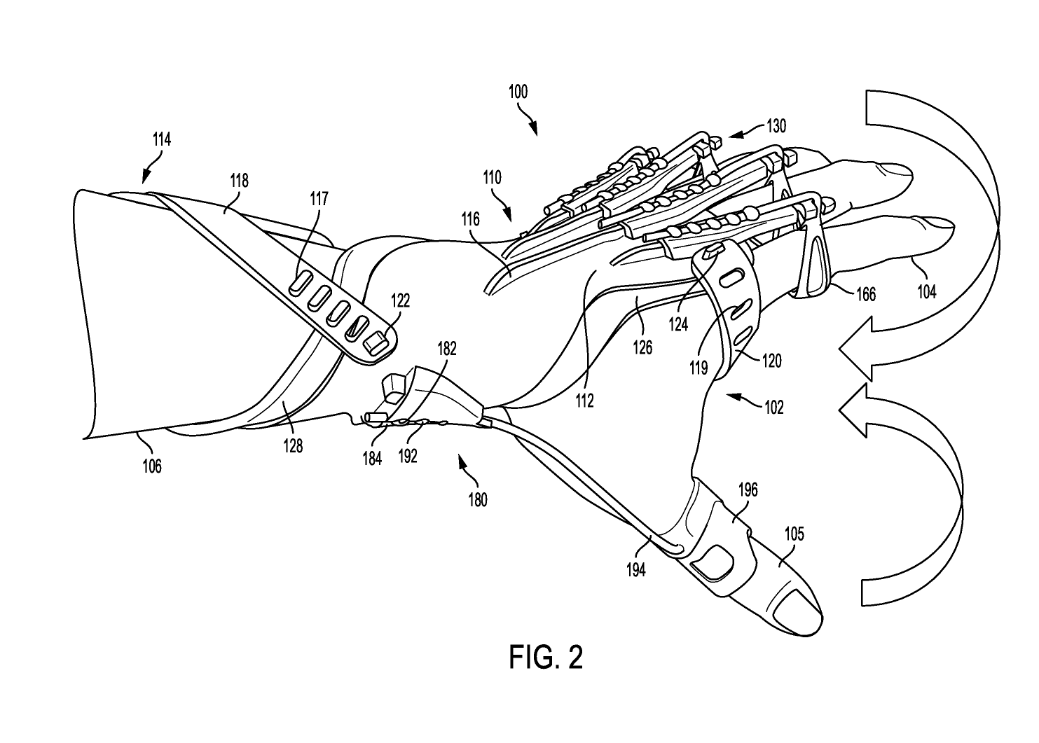

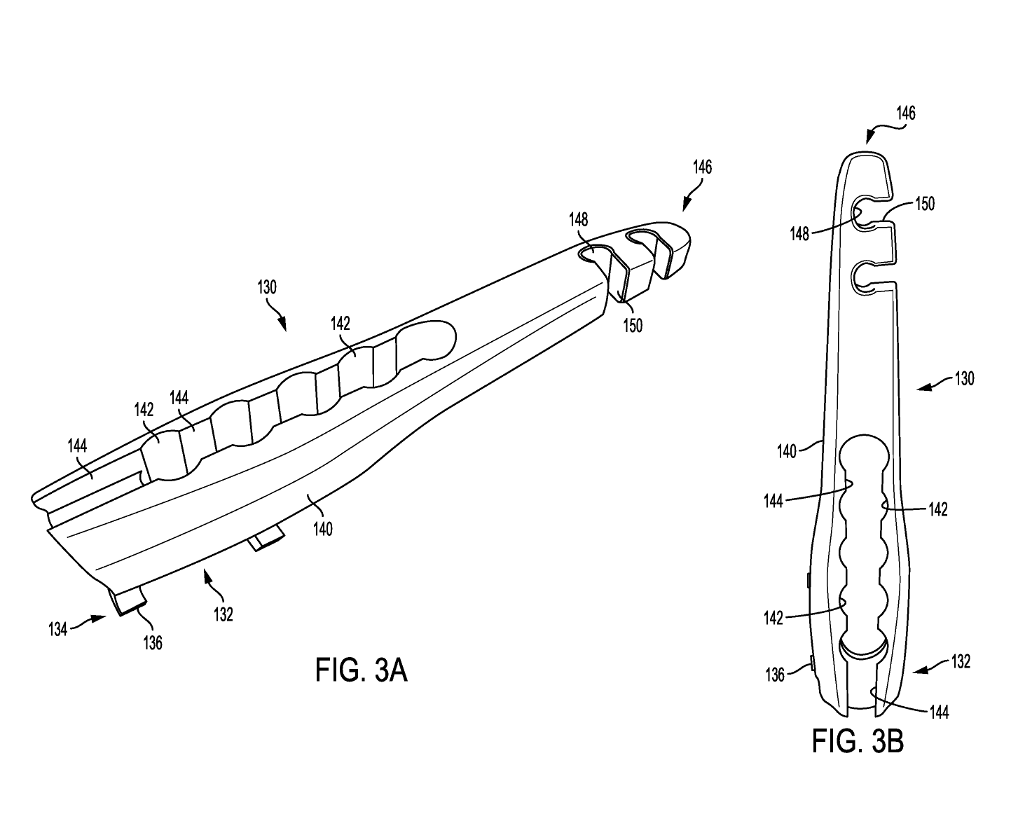

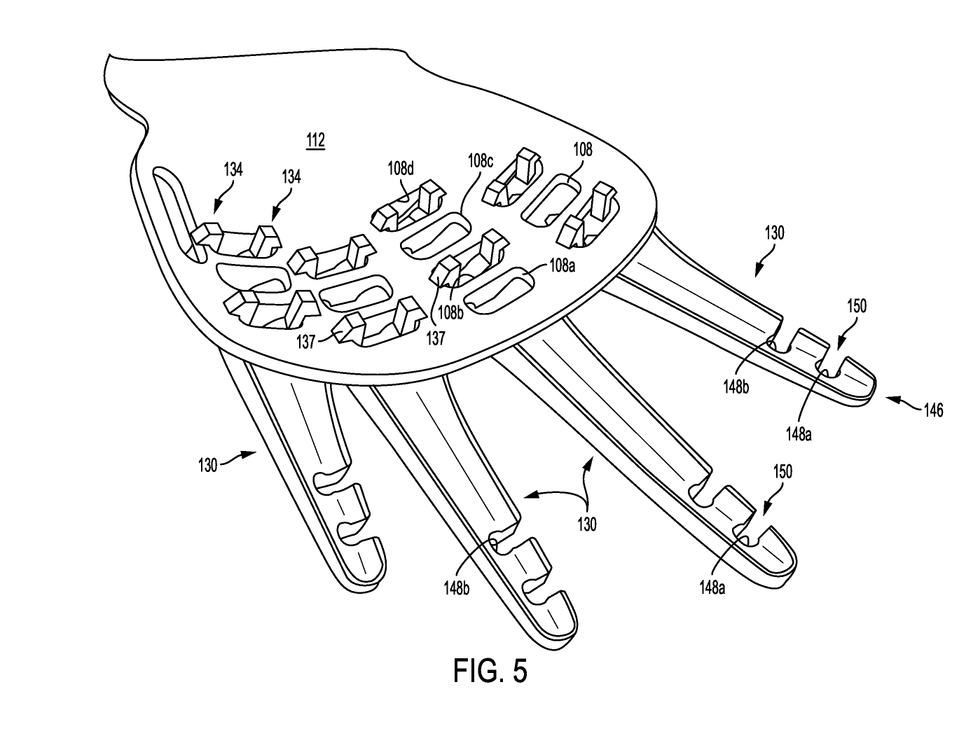

As best seen in FIGS. 1 and 2, dynamic hand splint 100 preferably includes an elongated finger strut 130 for each finger of the user's hand. Referring additionally to FIGS. 3A and 3B, each finger strut 130 includes a proximal end, or base 132, a distal end 146, and a body portion 140 extending therebetween. Each finger strut 130 is removably secured to platform 112 of hand support portion 110 by two pairs of opposed attachment legs 134 that are disposed on the bottom surface of its base 132. As best seen in FIG. 5, each attachment leg 134 of each pair includes an outwardly depending hook portion 136 disposed on its distal end. Each pair of attachment legs 134 is slidably received in a corresponding strut aperture 108 defined in platform 112 of hand support portion 110.

To mount each finger strut 130 to platform 112, each pair of attachment legs 134 is aligned with a corresponding strut aperture 108 on the top surface of platform 112. As each pair of attachment legs 134 is pushed inwardly through the corresponding strut aperture 108, the legs in each pair are cammed inwardly by the interaction of the cam surfaces 137 of the hook portions and the side walls of the corresponding strut aperture 108 until hook portions 136 are passed fully through platform 112. Once hook portions 136 are fully inserted in the corresponding strut aperture 108, the attachment legs 134 move outwardly to their original, undeflected positions so that hook portions 136 engage the underside of platform 112. As shown in FIG. 5, dependent upon the number of strut apertures 108 provided for each strut 130, the strut can be selectively mounted to the platform 112 so that the extent to which its distal end 146 extends outwardly therefrom is affected. For example, where four strut apertures 108 are provided, the pairs of attachment legs 134 may be placed in the first and third strut apertures 108a and 108c, or alternately in the second and fourth strut apertures 108b and 108d.

Referring again to FIGS. 3A and 3B, body portion 140 of each strut 130 defines a plurality of recesses 142 in its top surface. As well, a channel 144 is formed in the top surface of each body portion 140 so that channel 144 intersects, or passes through, each of the plurality of recesses 142. Each recess 142 is configured to selectively receive a node 164 of a corresponding elongated tensioner 160 in a press-fit, as discussed in greater detail below. Similarly, each channel 144 is configured to selectively receive a portion of a corresponding tensioner's body 162 in a press-fit. Preferably, a pair of apertures 148a and 148b is formed in the distal end 146 of each finger strut. Each aperture 148a, 148b includes an opening 150 in its side to allow a portion of a corresponding elongated tensioner 160 to be slid therethrough so as to be positioned in the desired aperture 148a, 148b, as shown in FIGS. 1 and 2. Having multiple apertures 148a, 148b in the distal end of each finger strut 130 allows the position of the tensioner with respect to the user's finger 104 to be adjusted as desired.

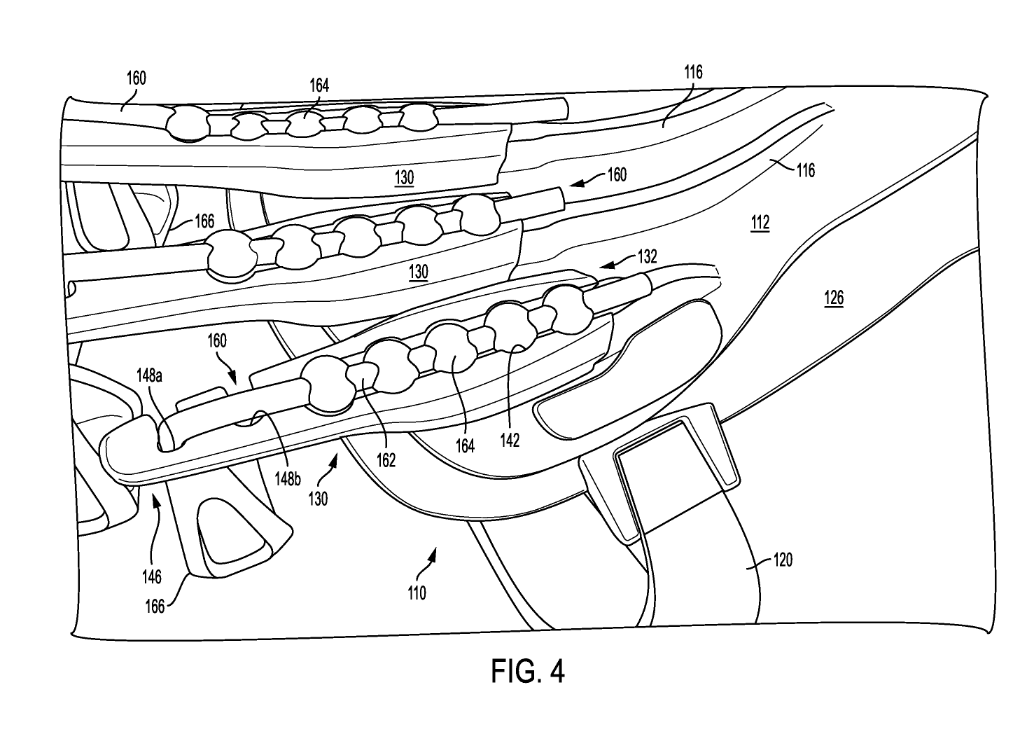

As best seen in FIG. 4, an elongated tensioner 160 is removably received in a corresponding finger strut 130 along its longitudinal center axis. Preferably, each tensioner 160 includes an elongated body portion 162 having a proximal end 161 and a distal end 167. A plurality of nodes 164 is disposed along proximal end 161 of body portion 162, and a cradle 166 is disposed at the distal end 167 of body portion 162. Preferably, each node 164 is equally spaced along body portion 162 and is configured to be slidably received in a recess 142 of the corresponding finger strut 130 in a press-fit. As shown, each node has a circular cross-section that is larger than the cross-section of the body portion 162 on which it is formed. Note, however, the shape of the cross-section of the nodes 164 may vary, such as, but not limited to, square, triangular, oval, diamond, etc. Preferably, each section of body portion 162 on which nodes 164 are disposed is also received in channel 144 of the corresponding finger strut 130 in a finger press-fit. Distal end 167 of the elongated tensioner 160 is passed through one of apertures 148a, 148b formed in its distal end 146 so that the corresponding cradle 166 of the elongated tensioner 160 may be disposed about finger 104 of the user. As previously noted, the position of cradle 166 relative to the user's finger is adjustable dependent upon which aperture 148a, 148b of tensioner 160 is utilized, as well as which recesses 142 of strut 130 are engaged by which nodes of the elongated tensioner 60.

Preferably, elongated tensioners 160 are molded of an elastic material, such as, but not limited to, silicone, rubber, etc. Such materials are selected so that each elongated tensioner 160 has sufficient flexural strength to resist, but not prevent, the bending of finger 104 that is received within the corresponding cradle 166. As such, when the finger is flexed from an extended position (FIG. 2) toward a flexed position (not shown), body 162 of tensioner 160 is elongated with the finger such that the finger is urged toward the extended position by the tensioner.

Referring again to FIGS. 1 and 2, in addition to the plurality of finger struts 130, hand splint 100 also includes a thumb strut 180 that is preferably secured to hand support section 110 in the same manner as the finger struts. As well, thumb strut 180 includes a plurality of recesses 182 disposed along a channel 184 formed in its upper surface. Recesses 182 and channel 184 are configured to releasably receive nodes 192 and elongated body 194 of elongated tensioner 190 in a press-fit, the elongated tensioner being releasably attached to a user's thumb 105. As shown, a collar 196 is disposed on the distal end of elongated tensioner 190, with collar 196 being configured to slidably receive the user's thumb 105 therein. Preferably, thumb tensioner 190 is comprised of the same elasticized material as used for finger tensioners 130.

In the embodiment shown, a covering 126 for the dorsum of the hand is integrally formed with a covering 128 for the forearm. In alternate embodiments, the hand and forearm covering 126 and 128 may be independently formed. In those embodiments, in which platform 112 is manufactured in an injection molding process, an area of hooks (not shown) is formed as an integral part of platform 112 during the molding process. The area of hooks preferably is adapted to attach to loops on at least a portion of the dorsum covering 126 in conventional hook-and-loop attachment, such as exemplified in VELCRO®-type attachments. This arrangement provides for secure placement of the components of the dynamic hand splint 100 during donning and use. As best seen in FIG. 1, forearm covering 128 extends rearwardly from the dorsum covering 126. Forearm covering 128 is configured to cushion the user's forearm 106 from the forearm support section 114 during use. The forearm covering 128 may also be secured to the forearm support section 114 by hook-and-loop attachment. Note, also, hook-and-loop type attachments may be used to selectively secure both wrist strap 118 and palm strap 120 when donning the splint, rather than attachment posts 122 and 124. Although the user's palm is left largely exposed in the present embodiment for comfort and ease of donning, in alternate embodiments a covering for the user's palm can be provided to make the dynamic hand splint more glove-like.

Referring now to FIGS. 6A, 6B and 6C, an alternate embodiment of a dynamic splint 200 in accordance with the present disclosure is illustrated. As shown in FIG. 7, dynamic splint 200 is configured for use in rehabilitating a user's foot 202. The present embodiment includes a cuff portion 210, a pair of platforms 212 affixed thereto, and a pair of anchor fittings 214 that is secured to a shoe 203 worn on the user's foot 202. Preferably, cuff portion 210 is positioned around the lower leg 204 of the user in the vicinity of the user's ankle, and secured thereto with a strap 218. Strap 218 includes a proximal end 217 that is affixed to cuff portion 210, and a distal end including a plurality of apertures 219 that are selectively securable to an attachment post 222, which is also disposed on cuff portion 210. As such, cuff portion 210 may be comfortably secured to variously sized lower legs 204.

As best seen in FIGS. 6C and 7, a pair of elongated tensioners 260 extend between cuff portion 210 and anchor fittings 214 of dynamic foot splint 200. Each elongated tensioner 260 includes a plurality of nodes 264 spaced evenly along its elongated body on its proximal end, and an anchor 266 disposed on its distal end. Each anchor 266 is configured to be received in an aperture 216 of a corresponding anchor fitting 214. As shown, anchor fittings 214 are unitarily formed and are affixed to shoe 203 utilizing its laces 207. Alternately, each anchor fitting 214 may have a threaded stem (not shown) that is passed through one of the shoes' lacing holes and engaged with a correspondingly threaded bore (not shown) on the inside of the shoe. In this manner, each anchor fitting 214 may be affixed to the user's shoe 203 independently of the other anchor fitting 214. The angle of the user's foot 202 relative to the user's lower leg 204 may be adjusted based on positioning of the nodes 264 of each elongated tensioner 260 within the plurality of recesses 242 formed in the corresponding platform 214. Similarly to the previously discussed dynamic hand splint 100, recesses 242 are formed along a channel 244 that is configured to receive the body of the corresponding elongated tensioner 260 in the press-fit. As well, elongated tensioners 260 are preferably formed of an elasticized material such as, but not limited to, silicone, rubber, etc.

In view of the foregoing, it will be appreciated that several preferred embodiments of dynamic hand splints 100 and 200 of the present invention have been disclosed and described in detail with reference to the drawings. Furthermore, other embodiments having alternative or equivalent features also have been and are disclosed, and equally are within the scope of the present invention. For instance, areas having loops and areas having hooks may be reversed in the aforementioned embodiments. Furthermore, while tensioners have been disclosed as extending along the dorsum of a respective digit by way of the struts, the tensioners may extend instead along a side of the respective digit.

While one or more preferred embodiments of the invention are described above, it should be appreciated by those skilled in the art that various modifications and variations can be made in the present invention without departing from the scope and spirit thereof. It is intended that the present invention cover such modifications and variations as come within the scope and spirit of the appended claims and their equivalents.

1. A dynamic splint for use with a hand of a user, comprising:
a hand support section adapted to be releasably secured to the hand of the user;
at least one strut including a base portion disposed adjacent the hand support section and a body portion extending outwardly therefrom to a distal end, the body portion defining at least one recess disposed between its proximal end and its distal end; and
at least one tensioner including a proximal end, a distal end configured to be secured to a digit of the user's hand, an elongated body extending therebetween, and at least one node disposed on the elongated body,
wherein the at least one node is selectively received in the at least one recess in a press-fit, and the elongated body of the at least one tensioner has flexural strength to resist bending of the digit secured to the distal end of the at least one tensioner when the digit is flexed from an extended position toward a flexed position.

2. The splint of claim 1, wherein the at least one strut further defines a channel extending along its body portion, wherein the channel intersects the at least one recess.

3. The splint of claim 2, wherein:
the at least one recess of the body portion of the at least one strut further comprises a plurality of recesses, and
the at least one node of the at least one tensioner further comprises a plurality of nodes.

4. The splint of claim 3, wherein the nodes of the at least one tensioner are evenly spaced along its elongated body.

5. The splint of claim 2, wherein the cross-sectional area of the at least one node is greater than the cross-sectional area of the elongated body of the at least one tensioner when taken in a plane transverse to a longitudinal center axis of the elongated body.

6. The splint of claim 5, wherein a cross-sectional shape of the at least one node is one of circular and oval.

7. The splint of claim 1, wherein the at least one tensioner further comprises lanyard extending therefrom that is configured to be disposed around the digit of the user's hand.

8. The splint of claim 1, further comprising a forearm support section configured to be releasably attached to a forearm of the user, wherein the hand support section is rigidly secured to the forearm support section.

9. The splint of claim 1, wherein when the digit is flexed from the extended position to the flexed position, the elongated body of the at least one tensioner is elongated with the digit and the digit is urged by the at least one tensioner toward the extended position.

10. A dynamic splint for use with an extremity of a user, comprising:
a support section configured to be releasably secured to a first portion of the extremity of the user, the support section defining at least one recess therein; and
at least one tensioner including a proximal end, a distal end configured to be secured to a second portion of the extremity of the user, an elongated body extending therebetween, and at least one node disposed on the elongated body,
wherein the at least one node is selectively received in the at least one recess in a press-fit, and the elongated body of the at least one tensioner has flexural strength to resist bending of the second portion of the extremity secured to the distal end of the at least one tensioner relative to the first portion of the extremity.

11. The splint of claim 10, wherein the support section further defines a channel extending inwardly from its outer surface, wherein the channel intersects the at least one recess.

12. The splint of claim 11, wherein the cross-sectional area of the at least one node is greater than the cross-sectional area of the elongated body of the at least one tensioner when taken in a plane transverse to a longitudinal center axis of the elongated body.

13. The splint of claim 11, wherein:
the at least one recess of the support section further comprises a plurality of recesses, and
the at least one node of the at least one tensioner further comprises a plurality of nodes.

14. The splint of claim 13, wherein the nodes of the at least one tensioner are evenly spaced along its elongated body.

15. The splint of claim 10, further comprising an anchor fitting configured to be releasably secured to the second portion of the extremity of the user, wherein the distal end of the at least one tensioner is secured to the anchor fitting.

16. The splint of claim 10, wherein the first portion of the extremity of the user is a lower leg, the second portion of the extremity of the user is a foot, and the support section further comprises a cuff that is configured to be disposed around the lower leg above an ankle of the user.

17. The splint of claim 11, wherein the support section further comprises at least one strut extending outwardly therefrom, the at least one strut including a proximal end adjacent the support section, a distal end, and a body portion extending therebetween, wherein the channel extends along the body portion, and wherein the first portion of the extremity of the user is a hand, and the second portion of the extremity is a digit of the hand.

18. The splint of claim 17, wherein the at least one tensioner further comprises lanyard extending therefrom that is configured to be disposed around the digit of the user's hand.

19. The splint of claim 17, further comprising a forearm support section configured to be releasably attached to a forearm of the user, wherein the support section is rigidly secured to the forearm support section.

20. A dynamic splint for use with a hand of a user, comprising:
a hand support section adapted to be releasably secured to the hand of the user;
at least one strut including a base portion disposed adjacent the hand support section and a body portion extending outwardly therefrom to a distal end, the body portion defining a channel extending along its body portion; and
at least one tensioner including a proximal end, a distal end configured to be secured to a digit of the user's hand, and an elongated body extending therebetween,
wherein a portion of the at least one tensioner is selectively received in the channel in a press-fit, and the elongated body of the at least one tensioner has flexural strength to resist bending of the digit secured to the distal end of the at least one tensioner when the digit is flexed from an extended position toward a flexed position.

21. The splint of claim 20, wherein:
the at least on strut further defines at least one recess disposed along the channel,
the at least one tensioner further comprises at least one node disposed along its elongated body, and
the at least one node is selectively received in the at least one recess in a press-fit.

22. The splint of claim 20, wherein when the digit is flexed from the extended position to the flexed position, the elongated body of the at least one tensioner is elongated with the digit and the digit is urged by the at least one tensioner toward the extended position.
